(19) United States
(12) Patent Application Publication
(10) Pub. No.: US 2008/0071370 A1
(43) Pub. Date: Mar. 20, 2008

(54) Reusable breast cover and nipple prosthesis

(76) Inventor: Luis A. Vinas, Ocean Ridge, FL (US)

Correspondence Address: SIMPSON & SIMPSON, PLLC, 5555 MAIN STREET, WILLIAMSVILLE, NY, 14221-5406, US

(21) Appl. No.: 11/523,219

(22) Filed: Sep. 18, 2006

(51) Int. Cl. A61F 2/52

(52) U.S. Cl. 623/7; 450/81

(57) ABSTRACT

The present invention is a covering for the nipple/areola area of the human breast. In one embodiment, the invention is a cover for the nipple and areola. In a second embodiment, the covering is a nipple prosthesis including an artificial nipple and areola. The covering is preferably fabricated from silicone having self adhering properties that enable the covering to be worn at the end of the breast without additional adhesive. As a nipple cover, the covering includes a concavity placed over the nipple to accommodate the nipple when the covering is worn. As a nipple prosthesis, in one embodiment, the covering includes a pocket or bubble positioned directly under the nipple portion of the prosthesis. After placing the prosthesis on the breast, the air in the pocket and or concavity is released to form a vacuum to allow the prosthesis to be held on the breast without the use of additional adhesive material.

### FIELD OF THE INVENTION

The present invention relates generally to the field of prostheses, more specifically to the field of breast prostheses, and still more specifically to the field of breast covers and nipple prostheses.

### BACKGROUND OF THE INVENTION

After breast surgery, such as partial or complete mastectomies, women are often faced with additional surgeries in the form of reconstructive procedures. Included in reconstructive surgery is the use of breast prostheses that attempt to impart the natural look that provides assurance to women as they proceed through a surgical program.

Problems exist with breast prostheses found in the prior art in that they often give an unnatural appearance to the bust line. In addition, prostheses are often made from materials that are stiff or hard and uncomfortable to wear for prolonged periods of time. Moreover, in the case of temporary, reusable prostheses, adhesives, glues, and/or tapes are often required to be reapplied to a reusable prosthesis which can lead to skin irritation and a lack of comfort.

U.S. Pat. No. 5,171,321 to Davis discloses a nipple prosthesis fabricated from polyvinylchloride, silicone, or other resilient materials and molded into the shape of a human nipple and areola. The '321 patent discloses the need for an adhesive on the back (contact) surface of the prosthesis. U.S. Patent Application Publication No. 2004/0010311 to Reynolds, et al. discloses an integrated nipple and areola prosthesis used in conjunction with a breast prosthesis. The prosthesis disclosed in the '311 publication comprises zones of cured gel that are built onto one another to form the shape of the nipple and surrounding areola. It is designed to become a permanent component of an external breast prosthesis.

U.S. Patent Application Publication No. 2004/0143325 to Holmes discloses an artificial nipple prosthetic device attachable to a human breast that has undergone breast reconstruction surgery. The prosthesis has a concave shape with a layer of water—resistant adhesive used to hold the prosthesis on to the breast. U.S. Pat. No. 6,497,609 to Cobbs discloses a device placed between a breast and an article of clothing that covers the breast. The device is designed to enhance the appearance of the breast and nipple. Although the '609 device does not utilize an adhesive, it explicitly requires that the clothing conform closely to the shape of the breast in order to hold the device in position. In addition, in contrast to other prosthetic devices, the '609 device is sized to cover a major portion of the breast, not just the nipple-areola area.

A related problem exists in which a woman may want to cover one or both breasts without revealing the outline of a nipple. Even though the user of such a pad or cover may want to conceal the outline of the nipple, the same requirements regarding materials and comfort remain. Typical of the prior art is U.S. Pat. No. 6,200,195 to Furuno, et al. which discloses an adhesive pad that can be molded to the shape of a particular body part such as an elbow, knee, or breast. Although it may be molded to cover a breast and conceal the nipple outline, it relies on an adhesive to hold the pad in place on the particular body part, including the breast. Therefore, it makes no disclosure or suggestion of a device or method of wearing a breast cover or pad without using an adhesive that may irritate the skin.

Therefore, there exists a need in the field for a temporary nipple/areola prosthesis that does not require an adhesive, provides a look and feel that is similar to the appearance and feel of a natural human breast, is comfortable to wear for prolonged periods, and can be worn with any type of clothing.

### SUMMARY OF THE INVENTION

The present invention broadly comprises a temporary, reusable covering for a human breast that includes a device ranging in diameter or width from about 3 to about 9 cm. and having an outer surface and an inner surface with the device gradually increasing in thickness or height from less than 1 mm at the edge of the device to about 2 mm-1 cm toward the center of the device. The inner surface forms a generally concave arch or concavity extending from or close to the edge. The covering is fabricated from a form of silicone possessing self-adhering or sticky qualities that enables the cover to adhere to a forward end of a human breast. In an alternate embodiment, the inner surface may incorporate a layer of washable, reusable, sticky or adhesive silicone. The covering is temporary in that it is removable and may be used more than one time. In another embodiment, the present invention also includes a protrusion in the form of a human breast nipple formed from the outer surface and is configured so that the protrusion extends up to about 2.0 cm. as measured vertically from the edge of the prosthesis and is up to about 1.5 cm in diameter. In a more preferred embodiment of the invention, the covering also includes a suction bubble formed from the inner surface under the protrusion.

An object of the invention is to provide a temporary nipple cover that is both comfortable and reusable.

A second object of the invention is to supply a temporary nipple cover that adheres to the breast without the use of adhesives.

A third object of the invention is to present a temporary nipple cover that is similar in appearance and feel to a natural human breast.

Another object of the invention is to provide a temporary nipple prosthesis that is both comfortable and reusable.

An additional object of the invention is to supply a temporary nipple prosthesis that adheres to the breast without the use of adhesives.

A further object of the invention is to present a temporary nipple prosthesis that is similar in appearance and feel to the nipple of a natural human breast.

An added object of the invention is to provide a removable breast covering or areola/nipple covering capable of decoration and receiving ornamentation.

### BRIEF DESCRIPTION OF THE SEVERAL VIEWS OF THE DRAWINGS

The nature and mode of the operation of the present invention will now be more fully described in the following detailed description of the invention taken with the accompanying drawing Figures, in which:

FIG. 1 is a side perspective view of a pair of breasts for which use of the breast cover and nipple prosthesis of the present invention is appropriate;

FIG. 2 is a top view of the nipple prosthesis of the present invention;

FIG. 3 is a side view of the nipple prosthesis of the present invention;

FIG. 4a is a cross section of a small size nipple prosthesis of the present invention taken generally along line 4-4 in FIG. 2;

FIG. 4b is a cross section of a medium size nipple prosthesis of the present invention taken generally along line 4-4 in FIG. 2;

FIG. 4c is a cross section of a large size nipple prosthesis of the present invention taken generally along line 4-4 in FIG. 2;

FIG. 5 is a bottom view of the nipple prosthesis of the present invention;

FIG. 6 depicts the side perspective view of FIG. 1 showing the placement of the nipple prosthesis on the left and right breasts;

FIG. 7 shows the nipple prosthesis in place on the left breast with the nipple and areola of the prosthesis in a natural position when compared to the natural nipple and areola on the right breast;

FIG. 8 depicts the same side perspective view of FIG. 1 except both the right breast and left breast possess natural nipples and areolas;

FIG. 9 is a top view of the nipple cover of the present invention;

FIG. 10 is a side view of the nipple cover of the present invention;

FIG. 11a is a cross section of a small size of the nipple cover of the present invention taken generally along line 11-11 of FIG. 9;

FIG. 11b is a cross section of a medium size of the nipple cover of the present invention taken generally along line 11-11 of FIG. 9;

FIG. 11c is a cross section of a large size of the nipple cover of the present invention taken generally along line 11-11 of FIG. 9;

FIG. 12 is a bottom view of the nipple cover of the present invention;

FIG. 13 depicts the side perspective view seen in FIG. 8 in which the nipple cover is applied to the right breast and is aligned for placement over the nipple and areola of the left breast;

FIG. 14 shows each of the nipple/areolar regions covered by the nipple cover of the present invention;

FIG. 15 is a top perspective view of an alternate embodiment of the nipple prosthesis of the present invention in which the top surface includes a “rough surface” comprising pimples, ridges, grooves and/or other features commonly found on the surface of a natural breast, areola, and nipple;

FIG. 16a is a cross section of an alternate embodiment of a small size nipple prosthesis of the present invention taken generally along line 16-16 in FIG. 15;

FIG. 16b is a cross section of an alternate embodiment of a medium size nipple prosthesis of the present invention taken generally along line 16-16 in FIG. 15;

FIG. 16c is a cross section of an alternate embodiment of a large size nipple prosthesis of the present invention taken generally along line 16-16 in FIG. 15;

FIG. 17 is a top perspective view of an alternate embodiment of the breast cover of the present invention in which the top surface includes a “rough surface” comprising pimples, ridges, grooves and other features commonly found on the surface of a natural human breast;

FIG. 18a is a cross section of an alternate embodiment of a small size of the nipple cover of the present invention taken generally along line 18-18 of FIG. 17;

FIG. 18b is a cross section of an alternate embodiment of a medium size of the nipple cover of the present invention taken generally along line 18-18 of FIG. 1.7;

FIG. 18c is a cross section of an alternate embodiment of a large size of the nipple cover of the present invention taken generally along line 18-18 of FIG. 17; and;

FIG. 19 is a front view of two nipple prostheses of the present invention including embodiments of ornamentation and designs applied or attached to the prostheses.

### DETAILED DESCRIPTION OF THE INVENTION

At the outset, it should be appreciated that like drawing numbers on different drawing views identify identical structural elements of the invention.

While the present invention is described with respect to what is presently considered to be the preferred embodiments, it is understood that the invention is not limited to the disclosed embodiments. The present invention is intended to cover various modifications and equivalent arrangements included within the spirit and scope of the appended claims.

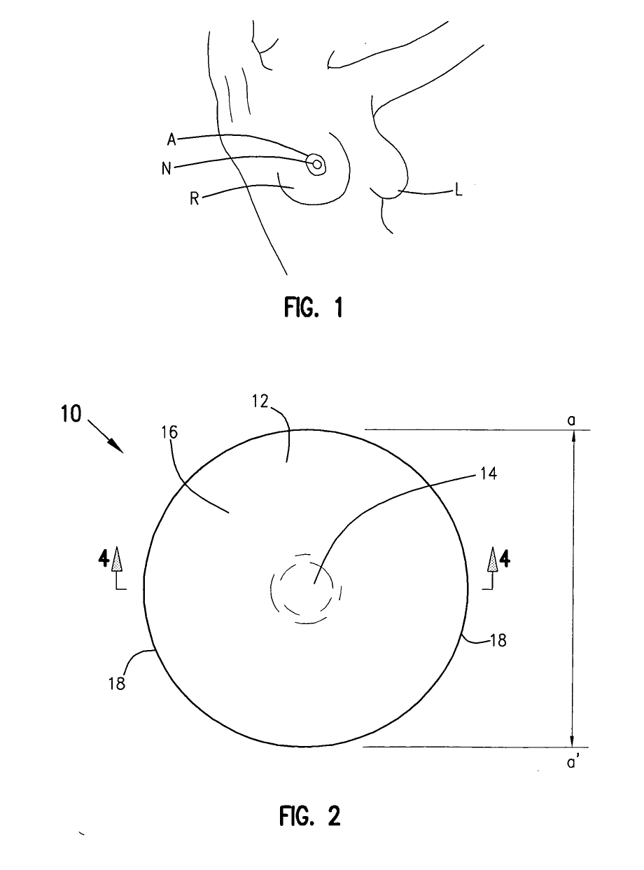

Adverting to the drawings, FIG. 1 is a side perspective view of a pair of breasts for which use of the breast cover and nipple prosthesis of the present invention is appropriate. Right breast R includes a nipple N and areola A while left breast L lacks a nipple and areola.

FIG. 2 is a top view of the nipple prosthesis 10 (“prosthesis 10”) of the present invention. Outer surface 16 of prosthesis 10 includes prosthetic nipple 14 (“nipple 14”) and areolar region 12 (“areola 12”) which surrounds nipple 14. Nipple 14 is in the form of a protrusion and is preferably located in the center of prosthesis 10 although it may be located in an off-center position if desired for an individual patient or particularly desired visual effect. Prosthesis 10 can range in size (width or diameter) from about 3-9 cm as measured by the gap between lines a and a′. Prosthesis 10 can be made from any number of plastics or fabrics designed to enable it to be washable and reusable. In a preferred embodiment, prosthesis 10 is fabricated from silicone in the form of liquid silicone rubber (LSR/LIM) or heat cured rubber (HCR/HCE) as this material provides both an appearance and feel nearly indistinguishable from the look and feel of a natural nipple and areola. Specific types of silicone for molding include, but are not limited to GE LIM 6010, GE CLS 2020 CLR, GE LIM 6030, GE LIM 6040, GE LIM 6050-D2, Wacker 3003/20, Wacker 3003/30, Shin Etsu KE1950-10 and Shin Etsu X-34-1784 and their equivalents. Although prosthesis 10 is shown as having a circular shape, it will be recognized by those having skill in the art that it may be formed into other shapes to fit a particular need including noncircular, nonsymmetrical, or irregular shapes. By irregular is meant a shape that is uneven.

FIG. 3 is a side view of prosthesis 10. Nipple 14 is seen as a protrusion formed from outer surface 16 and possessing sloping sides. In an alternate embodiment, nipple 14 may be cylindrically shaped with the cylindrical wall somewhat more perpendicular to outer surface 16. In one embodiment, nipple 14 may be up to approximately 2 cm in length as measured by the gap between arrows b and b′ in FIG. 3 and up to about 1.5 cm in diameter. In alternate embodiments, nipple 14 may be of any desired length as described below. Edge 18 of prosthesis 10 is a micro edge preferably less than 1 mm in thickness around the perimeter of prosthesis 10. Because edge 18 is a thin micro edge, it is difficult to exactly ascertain where the supporting breast and edge 18 meet when prosthesis 10 is applied to a breast. In a more preferred embodiment, edge 18 measures about 0.38 mm in thickness. This advantage provides a more natural look and feel to prosthesis 10, especially at edge 18 where it intersects with the natural breast giving prosthesis 10 the appearance and feel of a natural nipple and areola.

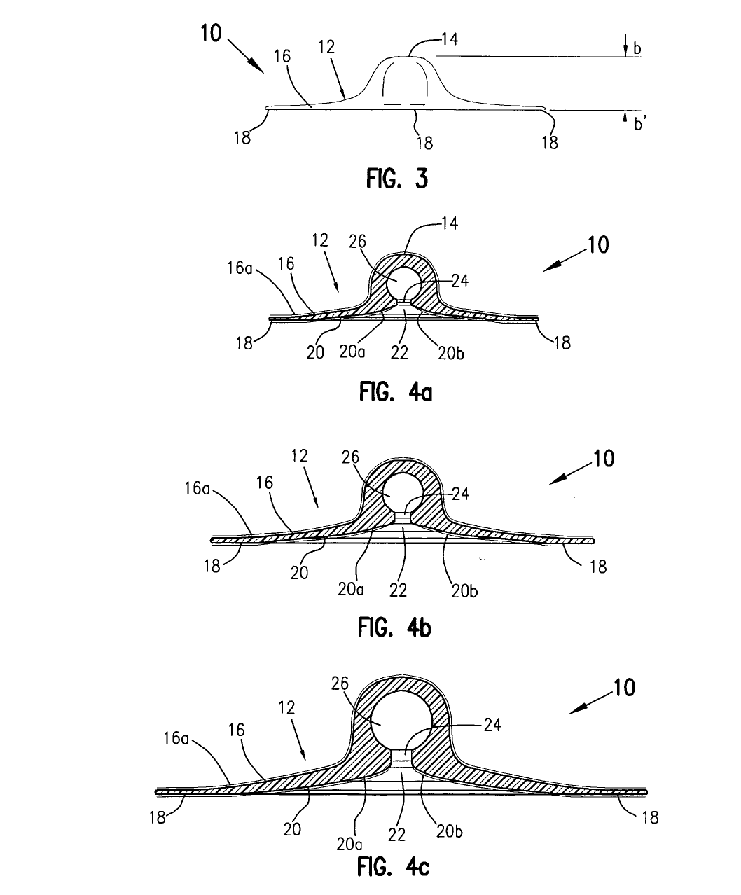

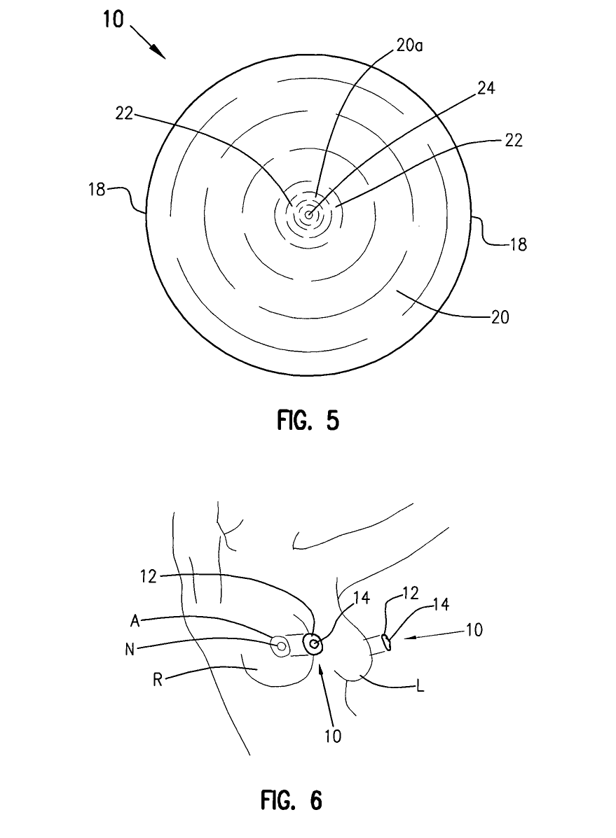

FIGS. 4a-c are cross sectional views of different sizes, small, medium, and large, respectively, of prosthesis 10 taken generally along line 4-4 in FIG. 2. In all three sizes, inner surface 20 is seen extending from the perimeter at edge 18 toward the center to form to center concavity 22. Concavity 22 is in the general shape of a concave arch formed from inner surface 20. Surface 20a is a continuation of inner surface 20 and forms the bottom surface of nipple 14 and the upper wall of concavity 22. In one embodiment, adhesive layer 20b (described below) is applied to inner surface 20 and surface 20a. The center of concavity 22 is preferably positioned over the position on the breast where the natural nipple would be. It will be recognized that concavity 22 of prosthesis 10 may also be paced over the natural nipple N of a human breast. This is advantageous if the user desires to maintain a similar appearance for both breasts. In the embodiment shown, passage 24 is positioned between concavity 22 and suction bubble 26 (“bubble 26”). In one embodiment, passage 24 ranges from about 1.7 to 3.2 mm in width and bubble 26 has a spherical diameter ranging from about 3-9 mm. The total height or thickness of prosthesis 10 ranges from 10-17 mm as measured from the vertical height between edge 18 and the peak of nipple 14 as represented by the gap between lines b and b′ in FIG. 3, while the diameter ranges from about 40 to about 70 mm. FIGS. 4a-c all depict the preferred embodiment in which prosthesis 10 gradually increases in thickness from the perimeter defined by edge 18 to nipple 14 (preferably located at the center of prosthesis 10). The diameter of the small, medium, and large prosthesis 10 is about 40 mm, 60 mm and 80 mm, respectively. In all three sizes, edge 18 measures about 0.38 mm in thickness. Persons of skill in the art will recognize that although three specific sizes of prosthesis 10 are shown and described, other embodiments of prosthesis 10 may have different diameters, heights, thicknesses, and shapes incorporated in the components of prosthesis 10. FIGS. 4a-c also show optional smooth layer 16a applied to outer surface 16. Both layers 16a and 20b are shown in exaggerated form for clarity. Layer 16a is normally very thin to achieve the desired natural look and feel. FIG. 5 is a bottom view of prosthesis 10. Passage 24 is preferably positioned in the center of concavity 22.

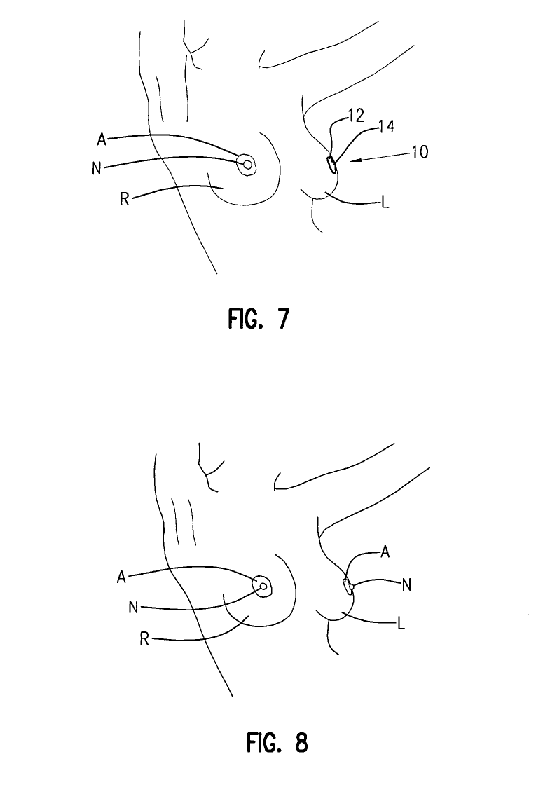

FIG. 6 depicts the side perspective view of FIG. 1 showing the placement of prosthesis 10 on left breast L and right breast R. It can be seen that a user may optionally place prosthesis 10 over a natural nipple N on right breast R as well as a breast without a nipple to attain the same or nearly the same look for both breasts L and R. FIG. 7 shows prosthesis 10 in place on left breast L with nipple 14 and areola 12 in a natural position when compared to nipple N and areola A on right breast R. The silicone material used to fabricate prosthesis 10 can possess a self adhering quality that enables prosthesis 10 to attach to left breast L without the use of additional, conventional fasteners such as tapes or glue. In the embodiment shown in FIGS. 4a-c, bubble 26 and/or concavity 22, which is filled with air when placed on breast L, may be “burped” similar to a plastic container top, such as a TUPPERWARE® container, to remove a quantity of air from bubble 26 and/or concavity 22 thereby creating a vacuum. This vacuum acts to hold prosthesis 10 in place without additional adhesive agents. In a preferred embodiment, a sticky silicone layer, such as Wacker SILGEL 612 OR GE 1408-02-18 seen as layer 20b in FIGS. 4a-c, may be applied to inner surface 20, to provide additional clinging capacity to hold prosthesis 10 in place, if desired. In an even more preferred embodiment, smooth layer 16a may be applied to outer surface 16 using a smooth silicone material such as GE Top Coat to provide a natural feel to outer surface 16 of prosthesis 10 whether or not sticky silicone layer 20b is applied to inner surfaces 20 and 20a.

In a second embodiment, the present invention is in the form of a pad or nipple cover 30 (“cover 30”). FIG. 8 depicts the same side perspective view of FIG. 1 except both right breast R and left breast L possess natural nipple N and areola A. Cover 30 may be used, for example, to provide padding for the nipple area of the natural breast, to present a desired visual appearance in which nipple N is not distinctly visible, and/or to cover breast, nipple, and/or areola scarring after surgery. It should be recognized that these examples are not to be considered to apply limits to the uses for cover 30. For example, cover 30 may be used if nipple N is not present. Persons of skill in the art will recognize that cover 30 may be fabricated from the same or equivalent materials as those used to produce prosthesis 10. In a preferred embodiment, cover 30 may include the application of the sticky silicones described above to inner surface 38 forming a layered composite, similar to that discussed above with prosthesis 10, to provide additional holding capacity to cover 30 when it is placed on a breast. In addition, a smooth silicone, such as GE Top Coat, may be applied to outer surface 34 of cover 30 whether or not adhesives are applied to inner surface 38. Consequently, cover 30 may possess the same self adhering qualities as prosthesis 10.

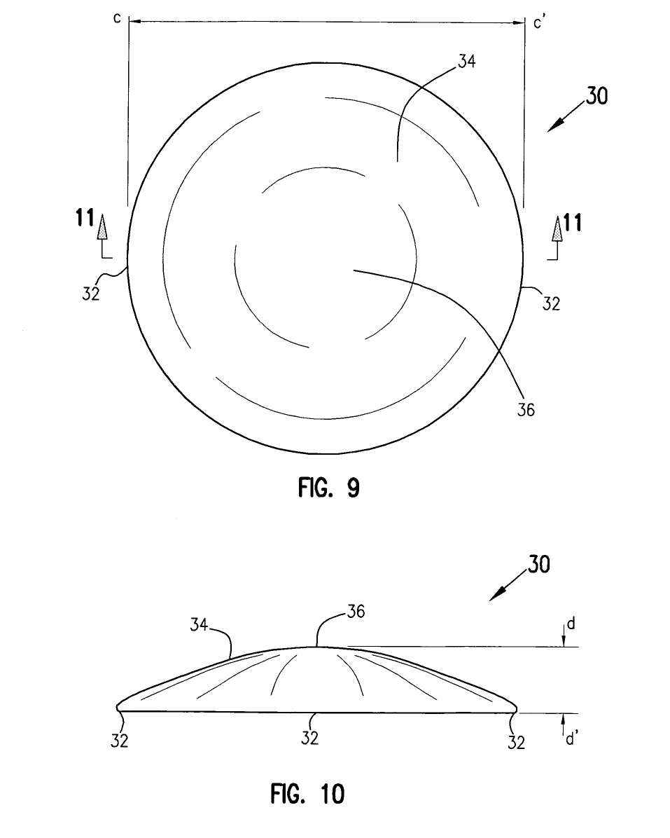

FIG. 9 is a top view of cover 30 with outer surface 34 showing micro edge 32 bounding the perimeter of cover 30. Similar to edge 18 of prosthesis 10, edge 32 is less than 1 mm in thickness. In a preferred embodiment, edge 32 is about 0.38 mm in width or thickness Cover 30 can range in diameter or width from 30-90 mm (3-9 cm) as measured by the gap between lines c and c′. In one embodiment, cover 30 may be 8-9 cm in diameter or width. Center 36 of cover 30 is also shown in FIG. 9. Although cover 30 is shown as having a circular shape, it will be recognized by those having skill in the art that it may be formed into other shapes to fit a particular need including noncircular, nonsymmetrical, or irregular shapes. By noncircular is meant a shape that does not form a circle at its edge or perimeter. Examples of a noncircular shape include but are not limited to, oblongs and rectangles. FIG. 10 is a side view of cover 30.

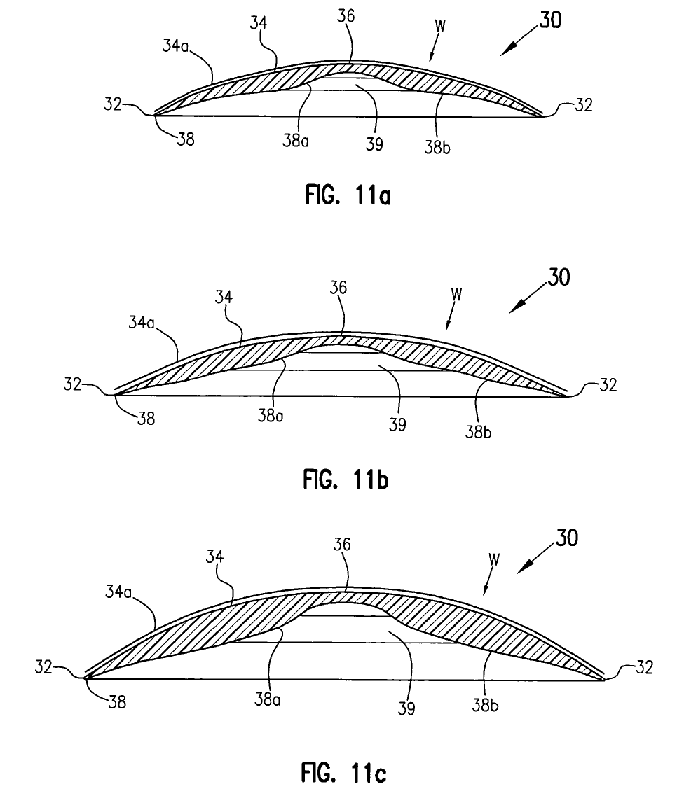

FIGS. 11a-c are cross sectional views of different sizes of cover 30 taken generally along line 11-11 of FIG. 9. Cover 30 increases in height from less than 1 mm at edge 32 to approximately 2 mm in the center as measured by the gap between lines d and d′ in FIG. 10. FIGS. 11a-c depict small, medium, and large sizes, respectively, of cover 30. In all three sizes, inner surface 38 is seen extending from edge 32 to center concavity 39. Surface 38a is a continuation of inner surface 38 and forms the upper wall of concavity 39. In the embodiments shown, surface 38a starts to extend from inner surface 38 at or near edge 32. Adhesive layer 38b is shown applied to inner surfaces 38 and 38a. Through the three sizes, the preferred diameter of cover 30 ranges from about 30 to about 90 mm. In an alternate embodiment, the height of cover 30 ranges from about 10 to about 17 mm measured as the vertical height from edge 32 to the highest point of cover 30 (the gap between lines d and d′ in FIG. 10). In the embodiments shown in FIGS. 11a-c, the highest point is generally in the center 36 of cover 30, but it will be understood that the highest point may be located elsewhere within the perimeter of cover 30. Throughout the different sizes of cover 30, edge 23 preferably measures about 0.38 mm in thickness. Smooth layer 34a is shown applied to outer surface 34. As in FIGS. 4a-c above, layers 34a and 38b are shown in exaggerated form for clarity. Layer 34a is normally very thin to achieve the desired natural look and feel. The circled areas W in each of FIGS. 11a-c represents the thickest portion of cover 30. Areas W range in thickness from 3-8 mm in the smallest to the largest sizes of cover 30, respectively.

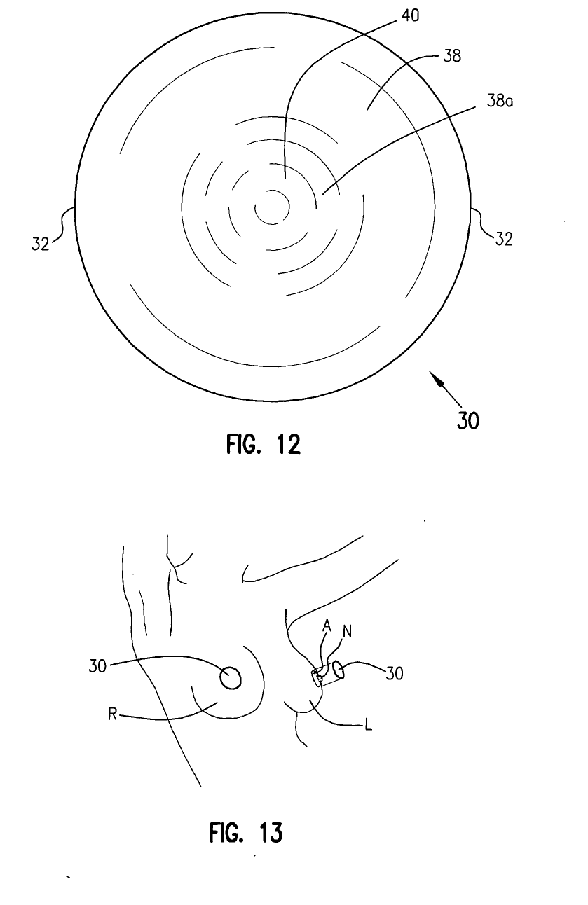

FIG. 12 is a bottom view of cover 30. Concavity 39 is again seen formed by inner surface 38a. Persons of skill in the art will recognize that although three specific sizes of cover 30 are show as described, other embodiments of cover 30 may have different diameters, heights, thicknesses, and shapes.

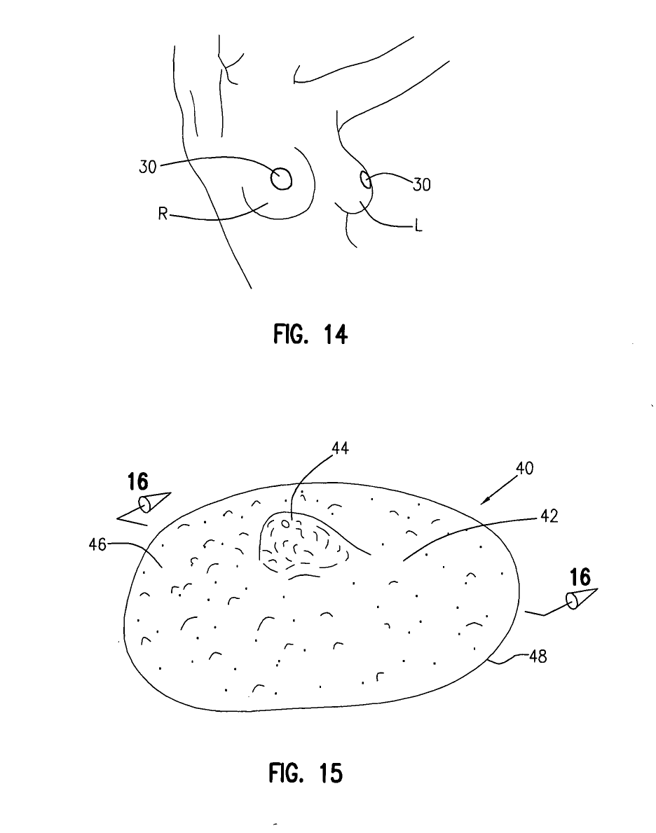

FIG. 13 depicts the side perspective view seen in FIG. 8 in which cover 30 is applied to right breast R and a second cover 30 is aligned to cover or conceal nipple N and areola A of left breast L. It can be seen in this view that covers 30 cover all of the nipple/areola regions of both breasts. FIG. 14 shows both nipple/areola regions covered by cover 30.

Similar to prosthesis 10, cover 30 is placed over each nipple/areola region and is held in place by the inherent adhesive quality of the silicone used to fabricate cover 30. Concavity 39 allows room for nipple N to fit comfortably under cover 30. In an alternate embodiment, cover 30 may be held comfortably in place by a sticky silicone layer 38b applied to inner surface 38 and/or 38a, similar to that used with inner surface 20 of prosthesis 10. In a preferred embodiment, a smooth silicone layer 34a is applied to outer surface 34 of cover 30. This smooth silicone serves to reduce friction between cover 30 and clothing as well as to produce a desirable tactile feel to cover 30. This layer may be made from GE Top Coat or equivalent material.

FIG. 15 is a top perspective view of an alternate embodiment of the nipple prosthesis, nipple prosthesis 40 (“prosthesis 40”), in which top surface 42 comprises a “rough surface” comprising pimples, ridges, grooves and/or other features commonly found on the surface of a natural breast, areola, and nipple. Nipple 44 is seen as a protrusion formed from outer surface 46 and possessing sloping sides. In an alternate embodiment, nipple 44 may be cylindrically shaped with the cylindrical wall somewhat more perpendicular to outer surface 46. In one embodiment, nipple 44 may be up to approximately 2 cm in length measured in a manner similar to that seen above using a gap analogous to the gap between arrows b and b′ in FIG. 3. The diameter of nipple 44 may be up to about 1.5 cm. In alternate embodiments, nipple 44 may be of any desired length as described below. Similar to prosthesis 10 and cover 30, prosthesis 40 includes edge 48 which is a micro edge preferably less than 1 mm in thickness around the perimeter of prosthesis 40. Because edge 48 is a thin micro edge, it is difficult to exactly ascertain where the supporting breast and edge 48 meet when prosthesis 40 is applied to a breast. In a more preferred embodiment, edge 48 measures about 0.38 mm in thickness. Areolar region 42 is seen on surface 46 of prosthesis 40.

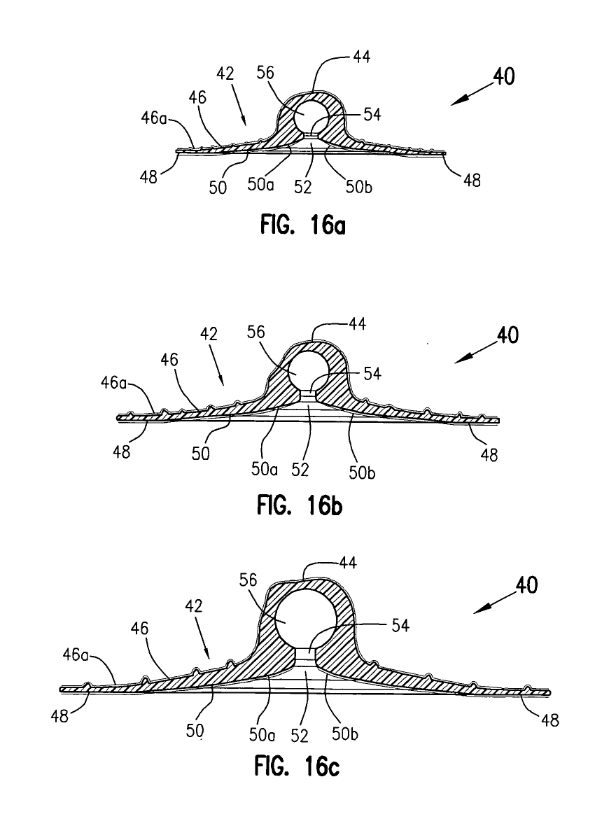

FIGS. 16a-c are cross sectional views of different sizes, small, medium, and large, respectively, of prosthesis 40 taken generally along line 16-16 in FIG. 15. In all three sizes, inner surface 50 is seen extending from the perimeter at edge 48 toward the center to form to center concavity 52. Concavity 52 is in the general shape of a concave arch formed from inner surface 50. Surface 50a is a continuation of inner surface 50 and forms the bottom surface of nipple 44 and the upper wall of concavity 52. In one embodiment, adhesive layer 50b (described below) is applied to inner surface 50 and surface 50a. Concavity 52 is preferably positioned over the position on the breast where the natural nipple would be. In the embodiment shown, passage 54 is positioned between concavity 52 and suction bubble 56 (“bubble 56”). In one embodiment, passage 54 ranges from about 1.7 to 3.2 mm in width and bubble 56 has a spherical diameter ranging from about 3-9 mm. The total height or thickness of prosthesis 40 ranges from 10-17 mm as measured from the vertical height between edge 48 and the peak of nipple 44 as analogous to the method described above utilizing the gap between lines b and b′ in FIG. 3. The diameter of nipple 44 ranges from about 40 to about 70 mm. FIGS. 16a-c all depict the preferred embodiment in which prosthesis 40 gradually increases in height from the perimeter defined by edge 48 to nipple 44 (preferably located at the center of prosthesis 40). The diameter of the small, medium, and large prostheses 40 is about 40 mm, 60 mm and 80 mm, respectively. In all three sizes, edge 48 preferably measures about 0.038 mm in thickness. Persons of skill in the art will recognize that although three specific sizes of prosthesis 40 are shown and described, other embodiments of prosthesis 40 may have different diameters, heights, thicknesses, and shapes in the components of prosthesis 40. A top coat 46a similar to that described above for prosthesis 10 and cover 30 may be applied to prosthesis 40. Similar to prosthesis 10 discussed above, it will be recognized that a user may optionally place prosthesis 40 over a natural nipple N on right breast R as well as a breast without a nipple to attain the same or nearly the same look for both breasts L and R.

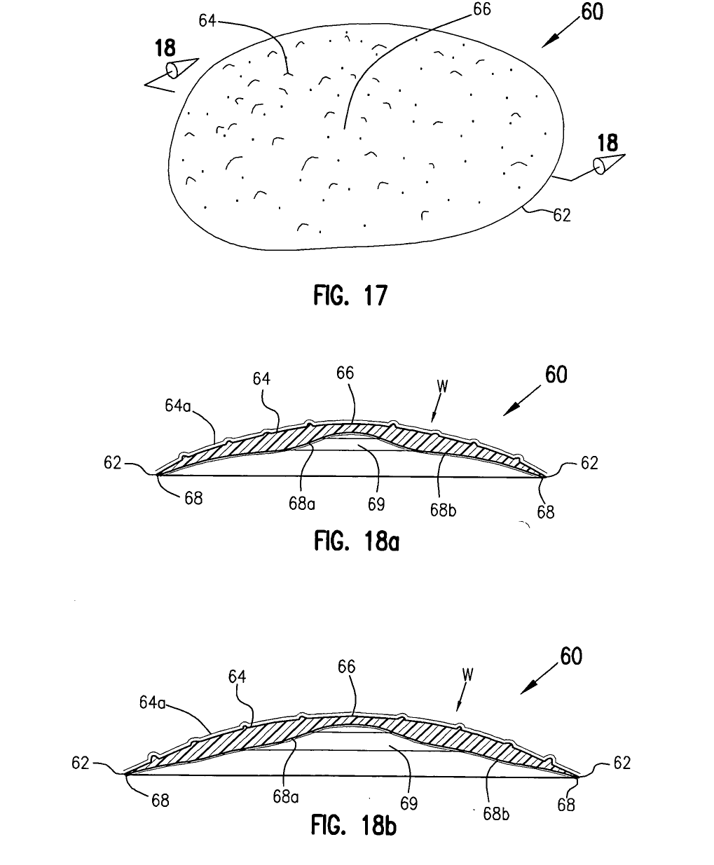

FIG. 17 is a top perspective view of breast cover 60 (“cover 50”) including an alternate embodiment in which outer surface 64 comprises a “rough surface” comprising pimples, ridges, grooves and/or other features commonly found on the surface of a natural breast. Cover 60 includes edge 62 which is a micro edge preferably less than 1 mm in thickness around the perimeter of cover 60. Because edge 62 is a thin micro edge, it is difficult to exactly ascertain where the supporting breast and edge 62 meet when cover 60 is applied to a breast. In a more preferred embodiment, edge 62 measures about 0.38 mm in thickness.

In one embodiment, cover 60 may be 8-9 cm in diameter or width. Center 66 of cover 60 is also shown in FIG. 17. Although cover 60 is shown as having a noncircular shape, it will be recognized by those having skill in the art that it may be formed into other shapes to fit a particular need including circular, nonsymmetrical, or irregular shapes. By noncircular is meant a shape whose perimeter does not form a circle. Examples of a noncircular shape include but are not limited to, oblongs and rectangles.

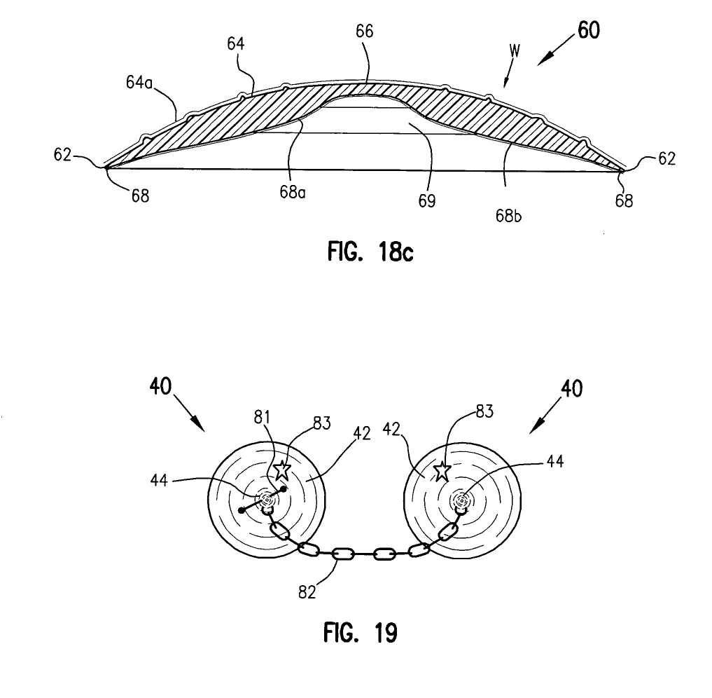

FIGS. 18a-c are cross sectional views of different sizes of cover 60 taken along line 18-18 in FIG. 17. Cover 60 increases in height from less than 1 mm at edge 62 to approximately 2 mm in the center as measured analogous to the gap between lines d and d′ in FIG. 10. FIGS. 18a-c depict small, medium, and large sizes, respectively, of cover 60. In all three sizes, inner surface 68 is seen extending from edge 62 to center concavity 69. Surface 68a is a continuation of inner surface 68 and forms the upper wall of concavity 69. In the embodiments shown, surface 68a starts to extend from inner surface 68 at or near edge 62. Adhesive layer 68b is shown applied to inner surfaces 68 and 68a. Through the three sizes, the preferred diameter of cover 60 ranges from about 30 to about 90 mm. In an alternate embodiment, the height of cover 60 ranges from about 10 to about 17 mm measured as the vertical height from edge 62 to the highest point of cover 60 (analogous to the gap between lines d and d′ in FIG. 10). In the embodiments shown in FIGS. 18a-c, the highest point is generally in the center 66 of cover 60, but it will be understood that the highest point may be located elsewhere within the perimeter of cover 60. Throughout the different sizes of cover 60, edge 62 measures about 0.38 mm in thickness. Adhesive layer 68b is shown applied to inner surface 68. Persons of skill in the art will recognize that although three specific sizes of cover 60 are show as described, other embodiments of cover 60 may have different diameters, heights, thicknesses, and shapes. A smooth top coat 64a similar to that applied to cover 30 above may also be applied to cover 60. The circled areas W in each of FIGS. 11a-c represents the thickest portion of cover 30. Areas W range in thickness from 3-8 mm in the smallest to the largest sizes of cover 60.

Both prostheses 10 and 40 and covers 30 and 60 may be fabricated by injection molding from liquid silicone rubber material. For prostheses 10 and 40, nipples 14 and 44, respectively, are formed as an integral part of prostheses 10 and 40, respectively, in the molding process. The liquid silicone rubber may be injected and cured in a mold that will range in temperature from 250-400° F. and the curing cycle time will normally range from 20 seconds to 2 minutes. In an alternate embodiment, a post cure process for the molded device(s) may be desired. The post cure is a secondary process in which the molded prostheses 10 and 40 or covers 30 and 60 are placed in an air circulating oven for 30 minutes to 8 hours with an oven temperature of 150-400° F. Post curing may be desirable to drive residual volatiles from the silicone material.

Alternative methods of molding prostheses 10 and 40 and covers 30 and 60 may include compression molding where the liquid silicone rubber or heat cured rubber is placed in compression molds and then molded in a compression vs. injection molding machine. Material is typically hand fed into these molds although automated feed dispensing equipment known to those skilled in the art may be used. Cycle times would normally range from 1-10 minutes and cure temperatures would be 250-400° F. Post cures may be applied as described above.

Molds used to make the body of both of the prostheses and both of the covers may be made of aluminum or steel. The molds may contain from one cavity up to several hundred cavities. After prostheses 10 and 40 or covers 30 and 60 are molded, they may then be transferred to a secondary processing area where silicone adhesive and/or top coat may be applied. The silicone adhesive may be Wacker SILGEL 612, GE 1408-02-18 or equivalent material. In either case the adhesive material is supplied in 1:1 ratio of A and B components which are hand mixed and kept refrigerated once mixed to preserve shelf life using methods known to those skilled in the art. The material is then applied to prostheses 10 and 40 and/or covers 30 and 60 using either a paint roller, paint brush or spray equipment. It is important that the entire surface be covered evenly with adhesive to ensure maximum tackiness. The molded prostheses 10 and 40 and/or covers 30 and 60 with the layer of silicone adhesive is then passed through a convection oven set between 200-400° F. for a time period of 5-20 minutes. [0038] Should a top coat layer be desired on the outer surface of molded prostheses 10 and 40 or covers 30 and 60, it may be applied before or after the above application of adhesive. The GE Top Coat material is also supplied in a 1:1 ratio (A and B components). This material is water like in viscosity and is also mixed by hand. The preferred method of application of the top coat is spraying but it may also be rolled or brushed on the desired molded product surface. This top coat layer is cured after application in a convection oven set at 200-400° F. for a time period of 2-10 minutes. Top coats are applied in a similar manner with smooth surface and “rough surface” embodiments.

Preferably, during fabrication of both prostheses 10 and 40 and covers 30 and 60, at least one pigment may be incorporated into the material to produce a color that blends with the natural breast/nipple/areola color(s) of the user. In an alternate embodiment, other colors may be used to produce a particular visual effect. Examples include, but are not limited to, fluorescent colors, “glow-in-the-dark” colors, pictures, drawings, simulated tattoos, and other designs limited only by the user's or manufacturer's imagination. Methods to incorporate pigments are well known to those skilled in the art.

Similarly, both prostheses 10 and 40 and covers 30 and 60 may be manufactured with orifices designed to receive ornaments, including but not limited to, jewelry or piercings or they may be pierced by the user to hold such ornaments. This provides the advantages of avoiding both the pain of applying jewelry and similar ornamentation to the natural breast and/or nipple and possible medical complications, including but not limited to infection, that may result from such piercing operations. FIG. 19 is a front view of two prostheses 10 which receive a piercing 81 and chain 82. Also seen is design 83 applied to the areola or outer surface of prosthesis 10. It will be recognized by those skilled in the art both that other articles may be applied to prostheses 40 and covers 30 and 60, as well as prosthesis 10.

Thus it is seen that the objects of the invention are efficiently obtained, although changes and modifications to the invention should be readily apparent to those having ordinary skill in the art, which changes would not depart from the spirit and scope of the invention as claimed.

### Parts List

A areola
L left breast
N nipple
R right breast
10 nipple prosthesis
12 areola region of prosthesis
14 nipple of prosthesis 10
16 nipple prosthesis top surface
18 nipple prosthesis micro edge
20 bottom surface
20a bottom concavity surface
22 concavity
24 passage
26 air bubble
30 nipple cover
32 nipple cover micro edge
34 nipple cover top surface
36 nipple cover center
38 nipple cover bottom surface
38a concavity surface
39 nipple cover concavity
40 nipple prosthesis (“rough surface”)
42 areola region of prosthesis 40
44 nipple of prosthesis 40
46 outer surface of prosthesis 40
48 micro edge of prosthesis 40
50 inner surface of prosthesis 40
50a concavity surface
52 concavity of prosthesis 40
54 passage of prosthesis 40
56 suction bubble of prosthesis 40
60 nipple cover (“rough surface”)
62 micro edge of cover 60
64 outer surface of cover 60
66 center or peak of cover 60
68 inner surface of cover 60
68a inner surface of concavity of cover 60
69 concavity of cover 60
81 piercing
82 chain
83 design

1. A temporary, reusable covering for a human breast comprising:
a device having a width of about 3-9 cm, said device having an outer surface and an inner surface, said device gradually increasing in height from less than 1 to about 2 mm at the edge of said device to about 2 mm to about 1 cm at the center of said device;
wherein said inner surface forms a generally concave arch extending from said perimeter toward said center of said device; and,
wherein said covering is fabricated from a self adhering silicone enabling said covering to removably adhere to said human breast.

2. The temporary, reusable covering for a human breast as recited in claim 1 wherein said edge is about 0.38 mm in thickness.

3. The temporary, reusable covering for a human breast as recited in claim 1 further comprising a layer of an adhesive silicone on said inner surface.

4. The temporary, reusable covering for a human breast as recited in claim 1 wherein said covering is circular and ranges from about 8-9 cm in diameter.

5. (canceled)

6. The temporary, reusable covering for a human breast as recited in claim 1 wherein said covering is noncircular in shape.

7. (canceled)

8. (canceled)

9. (canceled)

10. The covering for a human breast as recited in claim 1 further comprising a smooth silicone layer on said outer surface.

11. (canceled)

12. The covering for a human breast as recited in claim 1 wherein said outer surface is a rough surface including at least one of bumps, grooves, and ridges.

13. The covering for a human breast as recited in claim 1 further comprising at least one pigment incorporated into said silicone.

14. The covering for a human breast as recited in claim 1 further comprising ornamentation, at least one picture, and/or at least one design.

15. The covering for a human breast as recited in claim 1 wherein the maximum thickness between said outer surface and said inner surface ranges between about 3 to 8 mm.

16. A temporary, reusable covering for the alveolar/nipple region of a human breast comprising:
a device ranging in diameter having a width of about 3-9 cm, said device having a outer surface and a inner surface, said device gradually increasing in height from less than 1 mm to about 2 mm at the edge of said device to about 2 mm to about 1 cm at the center of said device;
a protrusion wherein said protrusion extends above said front surface;
wherein said inner surface forms a generally concave arch extending from or approximately from said edge toward said center of said device; and,
wherein said covering is fabricated from a self adhering silicone enabling said covering to adhere to said human breast.

17. The temporary, reusable covering for a human breast as recited in claim 16 wherein said edge is about 0.38 mm in thickness.

18. The temporary, reusable covering for the alveolar/nipple region of a human breast as recited in claim 16 further comprising a layer of an adhesive silicone on said inner surface.

19. The temporary, reusable covering for the alveolar/nipple region of a human breast as recited in claim 16 wherein said covering is round and ranges from about 8-9 cm in diameter.

20. The temporary, reusable covering for the alveolar/nipple region of a human breast as recited in claim 16 further comprising a suction bubble formed from said inner surface under said protrusion.

21. The temporary, reusable covering for the alveolar/nipple region of a human breast as recited in claim 16 wherein said covering is about 5-6 cm in width and ranges in height from about less than 1 mm at the edge of said covering to about 1 cm in height in the center of said covering.

22. The temporary, reusable covering for a human breast as recited in claim 16 wherein said protrusion extends up to about 2 cm above said front surface and is up to about 1.5 cm in diameter.

23. The temporary, reusable covering for the alveolar/nipple region of a human breast as recited in claim 16 further comprising a smooth silicone layer on said outer surface.

24. (canceled)

25. The temporary, reusable covering for the alveolar/nipple region of a human breast as recited in claim 16 wherein said outer surface is a rough surface including at least one of bumps, grooves, and ridges.

26. The temporary, reusable covering for the alveolar/nipple region of a human breast as recited in claim 16 further comprising at least one pigment incorporated into said silicone.

27. The temporary, reusable covering for the alveolar/nipple region of a human breast as recited in claim 16 further comprising ornamentation, at least one picture and/or at least one design.

28. (canceled)

29. (canceled)

30. (canceled)

31. (canceled)

32. (canceled)

33. (canceled)

34. (canceled)

35. (canceled)

36. (canceled)

37. (canceled)

38. (canceled)
